SEQUENCE LISTING (9 sequences)

SEQ ID NO: 1 - protein, 330 aa, Streptococcus pyogenes
NHKVHMHHHHHHADEQEEKAKVRTELIQELAQGLGGIEKKNFPTLGDEDLDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 2 - protein, 292 aa, Streptococcus pyogenes
NHKVHMHHHHHHDHTYMTKLLTYLQEREQAENSWRKRLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 3 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 4 - protein, 240 aa, Streptococcus pyogenes
GSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 5 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPAGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 6 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPQGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 7 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPAGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 8 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPSGPRGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

SEQ ID NO: 9 - protein, 267 aa, Streptococcus pyogenes
NHKVHMHHHHHHLLKGIQDHALDLVPRGSPGLPGPRGEQGPTGPTGPAGPRGLQGLQGLQGERGEQGPTGPAGPRGLQGERGEQGPTGLAGKAGEAGAKGETGPAGPQGPRGEQGPQGLPGKDGEAGAQGPAGPMGPAGERGEKGEPGTQGAKGDRGETGPVGPKGERGEAGPAGKDGERGPVGPAGKDGQNGQDGLPGKDGKDGQNGKDGLPGKDGKDGQNGKDGLPGKDGKDGQDGKDGLPGKDGKDGLPGKDGKDGQPGKPGKY

(19) United States
(12) Patent Application Publication
(10) Pub. No.: US 2025/0195715 A1
(43) Pub. Date: Jun. 19, 2025

(54) SPONGES BASED ON COLLAGEN-LIKE PROTEINS

(71) Applicant: Evonik Operations GmbH, Essen (DE)

(72) Inventors: Maren Jannasch, Darmstadt (DE); Sven Weber, Dossenheim (DE); Maria Montero Mirabet, Muehltal (DE)

(73) Assignee: Evonik Operations GmbH, Essen (DE)

(21) Appl. No.: 18/839,767

(22) PCT Filed: Feb. 10, 2023
(86) PCT No.: PCT/EP2023/053300

§ 371 Date: Aug. 20, 2024

(30) Foreign Application Priority Data
Feb. 25, 2022 (EP) 22158799.1

(51) Int. Cl. A61L 24/00; A61L 24/10

(52) U.S. Cl. CPC A61L 24/0036; A61L 24/102; A61L 2400/04

(57) ABSTRACT

A method for preparing a sponge based on collagen-like proteins starts with providing an aqueous solution having at least one collagen-like protein. A cross-linker reacts with the collagen-like protein to obtain a hydrogel and lyophilization leads to the sponge. The sponge obtained by the method shows increased performance in wound sealing, haemostasis, wound plugging, healing promotion, bone regeneration, cartilage repair, cell cultures, production of vegetarian or vegan meat or the absorption of biological fluids, like blood or wound exudate.

### FIELD OF THE INVENTION

The present invention relates to a method of preparing a sponge based on collagen-like proteins comprising the steps:
i) providing an aqueous solution comprising at least one collagen-like protein and optionally at least one additive;
ii) cross-linking the at least one collagen-like protein with at least one cross-linker via incubation to obtain a hydrogel;
iii) optionally washing the hydrogel with a buffer;
iv) performing a lyophilization step to obtain the sponge;
v) optionally adding at least one additive; and
vi) optionally sterilizing the obtained sponge.

Furthermore, the present invention pertains to a sponge obtained by the method according to the present invention and use of the sponge for wound sealing, haemostasis, wound plugging, healing promotion, bone regeneration, cartilage repair, cell cultures, production of vegetarian or vegan meat, the absorption of biological fluids, like blood or wound exudate.

### BACKGROUND

The object of the present invention was to provide alternatives to animal derived collagen sponges, which can be used in the medical field for example for wound sealing, haemostasis, wound plugging, healing promotion, bone regeneration, cartilage repair, cell cultures or the absorption of biological fluids, like blood or wound exudate. The alternative sponges should at least have similar characteristics, when applied in medical applications and in addition avoid challenges and issues of animal collagen derived sponges like allergies or intolerances.

Additionally, animal collagens exhibit poor solubility in water and form a viscous solution in acetic acid or hydrochloric acid. By first freeze drying the viscous solution and then cross-linking a freeze-dried material, a direct contact of the cross-linker solution to the protein network is achieved (solid-liquid stabilization). This procedure is faster than by diffusion of the cross-linker through a viscous solution. However, because dried proteins partially re-enter solution, the initial network structure of the freeze-dried material cannot be efficiently preserved. Finally, after cross-linking a freeze-dried material, the cross-linked product is again in wet state and needs a further freeze-drying step.

In this regard the inventors of the present invention found that the sponges according to the present invention based on collagen-like protein not only can overcome one or more of the above-mentioned issues, but the obtained sponges also surprisingly show improved form stability after being contacted with a liquid compared to sponges based on animal derived collagen. Furthermore, the sponges according to the present invention show improved stiffness, Young's modulus properties as well as open porosity. Moreover, the present process is as well improved in view of the “common” process for sponges derived from animal collagen by requiring less process steps.

### SUMMARY OF THE INVENTION

Therefore, in a first aspect, the present invention refers to a method of preparing a sponge based on collagen-like proteins comprising the steps:
i) providing an aqueous solution, comprising at least one collagen-like protein and optionally at least one additive;
ii) cross-linking the at least one collagen-like protein with at least one cross-linker via incubation to obtain a hydrogel;
iii) optionally washing the hydrogel with a buffer;
iv) performing a lyophilization step to obtain the sponge;
v) optionally adding at least one additive; and
vi) optionally sterilizing, preferably via plasma, gamma or UV treatment, the obtained sponge.

In a second aspect, the present invention pertains to a sponge obtained by the method according the present invention.

Finally, in a third aspect, the present invention refers to the use of the sponge according to the present invention for wound sealing, haemostasis, wound plugging, healing promotion, bone regeneration, cartilage repair, cell cultures, production of vegetarian or vegan meat, the absorption of biological fluids, like blood or wound exudate.

### DESCRIPTION OF THE FIGURES

FIG. 1 Fluid absorption was characterized for sponges made with different cross-linking technologies and concentrations. After incubation for 24 h in phosphate buffered saline, the fluid absorption was calculated by wet weight measurements relatively to dry weight. As a reference for animal-derived collagen, sponges made from rat tail collagen (5 mg/ml) were compared to sponges made from recombinant collagen-like protein (rCol; 10 mg/ml). All analysis were conducted in technical triplicates (n=3).

FIG. 2 Before and after submersion for 24 h in phosphate buffered saline, the diameter of sponges was assessed. Obtained values were applied to calculate the relative diameter change. Thereby, an evaluation of the swelling or the shrinkage of sponges made from collagen-like protein (rCol; 10, 20, and 40 mg/ml) and rat tail collagen (5 mg/ml) was feasible. Technical triplicates (n=3) were applied for all experimental groups.

FIG. 3 Pore sizes were measured by analysis of scanning electron microscope images, derived from cryo cross-sections. Dependent on the structure of the sponges and the resultant feasibility to measure sizes at least 8 to 27 pores (n≥8) were captured.

FIG. 4 Total porosity of sponges made from collagen-like protein (rCol; 10 and 40 mg/ml) or from rat tail collagen (5 mg/ml) was assessed by a liquid displacement method, employing ethanol. All experimental groups were analyzed in technical triplicates (n=3).

FIG. 5 Open porosity was quantified to characterize surface structure of sponges made from collagen-like protein (rCol; 10 and 40 mg/ml) or sponges made from rat tail collagen (5 mg/ml). The analysis was conducted in technical triplicates (n=3).

FIG. 6 Sponges made from collagen-like protein (rCol; 20 mg/ml) and a commercial reference Lyostypt (B. Braun) were compressed by a mechanical testing device. Recorded stress-strain correlations were applied to derive Young's modulus. Mean values of Young's modulus were derived from at least three independent compression cycles (n≥3).

FIG. 7 For an evaluation of Young's modulus under wetted conditions, respective sponges made from collagen-like protein (rCol, 20 mg/ml) were incubated for 24 h in phosphate buffered saline. Thereafter, the sponges were compressed to derive from stress-strain correlations the Young's modulus. Mean values were calculated from at least triplicates (n≥3).

FIG. 8 For a first comparison of collagen source to behavior, fluid absorption of sponges made with collagen-like protein (20 mg/ml) of Corynebacterium was assessed. Analysis was conducted with sponges made from 0.5 ml hydrogels. All calculations were derived from technical triplicates (n=3).

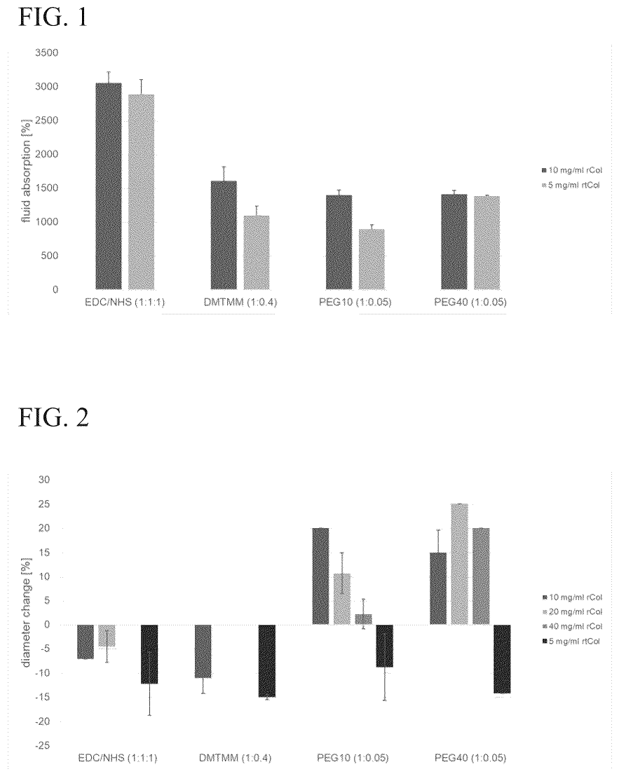

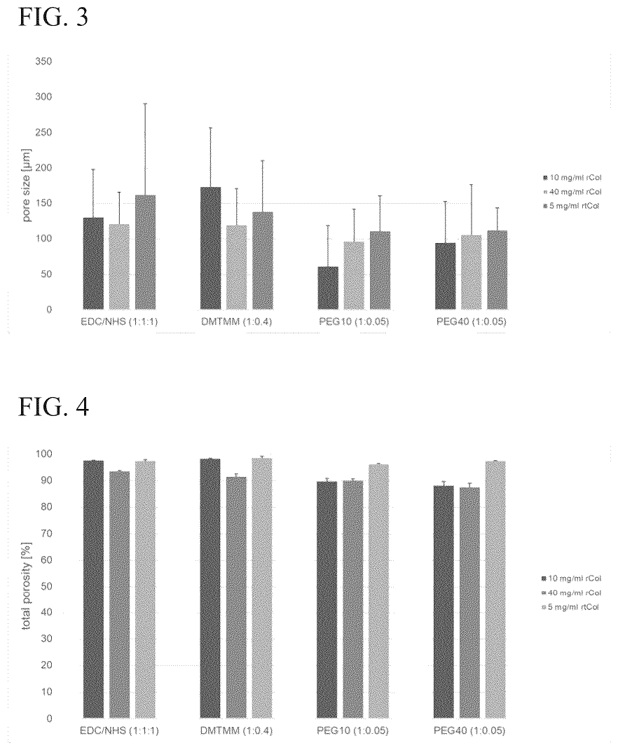

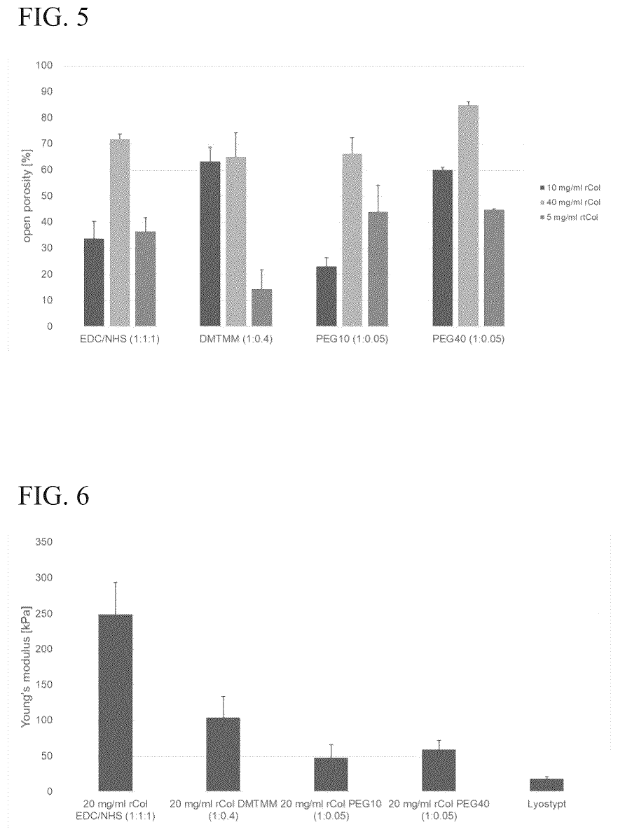

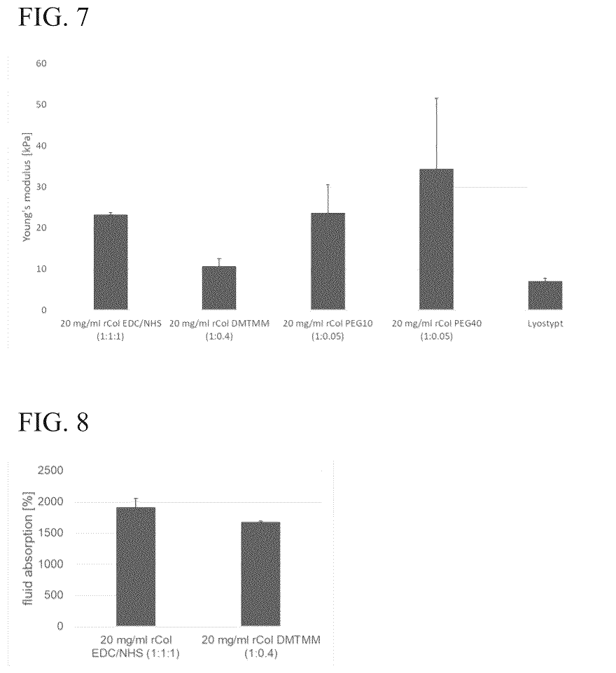

### DETAILED DESCRIPTION

Numerical ranges that are indicated in the format “from x to y” also include the stated values. If several preferred numerical ranges are indicated in this format, it is self-evident that all ranges that result from the combination of the various endpoints are also included.

“One or more”, as used herein, relates to at least one and comprises 1, 2, 3, 4, 5, 6, 7, 8, 9 or more of the referenced species. Similarly, “at least one” means one or more, i.e. 1, 2, 3, 4, 5, 6, 7, 8, 9 or more. “At least one”, as used herein in relation to any component, refers to the number of chemically different molecules, i.e. to the number of different types of the referenced species, but not to the total number of molecules. For example, “at least one surfactant” means that at least one type of molecule falling within the definition for a surfactant is used but that also two or more different types of surfactants falling within this definition can be present but does not mean that only one or more molecules of one type of surfactant are present.

All percentages given herein in relation to the compositions or formulations relate to wt.-% relative to the total weight of the respective composition, if not explicitly stated otherwise.

“Essentially free of” according to the present invention with regard to compounds means that the compound can only be present in an amount, which does not influence the characteristics of the composition, in particular the respective compound is present in less than 3 wt.-%, preferably 1 wt.-%, more preferably 0.01 wt.-%, based on the total weight of the composition or is not present at all.

The weight average molecular weight Mw and the number average molecular weight Mn can be determined by GPC employing polystyrene standards.

Therefore, in a first aspect, the present invention refers to a method of preparing a sponge based on collagen-like proteins comprising or consisting of the steps:
i) providing an aqueous solution, preferably having a pH value of 6 to 8, preferably 6.8 to 7.4, comprising at least one collagen-like protein and optionally at least one additive;
ii) cross-linking the at least one collagen-like protein with at least one cross-linker via incubation to obtain a hydrogel;
iii) optionally washing the hydrogel with a buffer;
iv) performing a lyophilization step to obtain the sponge;
v) optionally adding at least one additive; and
vi) optionally sterilizing, preferably via plasma, gamma or UV treatment, the obtained sponge.

In a second aspect, the present invention pertains to a sponge obtained by the method according the present invention.

Finally, in a third aspect, the present invention refers the use of the sponge according to the present invention for wound sealing, haemostasis, wound plugging, healing promotion, bone regeneration, cartilage repair, cell cultures, production of vegetarian or vegan meat, the absorption of biological fluids, like blood or wound exudate.

These and other aspects, embodiments, features, and advantages of the invention will become apparent to a person skilled in the art through the study of the following detailed description and claims. Any feature from one aspect of the invention can be used in any other aspect of the invention. Furthermore, it will readily be understood that the examples contained herein are intended to describe and illustrate the invention but not to limit the invention and that, in particular, the invention is not limited to these examples.

In the method at least one collagen-like protein is used. In general, all collagen-like proteins are suitable.

In a preferred embodiment of the present invention the collagen-like protein is a collagen-like protein from Streptococcus pyogenes, which is preferably the Scl2 protein from Streptococcus pyogenes.

Expression of collagen-like proteins have been attempted in several systems, including Escherichia coli and Saccharomyces cerevisiae.

In one embodiment the at least one collagen-like protein is a bacterial collagen-like protein, preferably produced by fermentation in Pichia, Brevibacillus, Bacillus, Escherichia or Corynebacterium, preferably Pichia pastoris, Brevibacillus choshinensis or Corynebacterium glutamicum.

In a preferred embodiment the collagen-like proteins may be expressed in Corynebacterium, preferably in Corynebacterium glutamicum.

One particularly suitable collagen-like protein is derivable from following polynucleotide.

A polynucleotide encoding an amino acid sequence that is at least ≥60%, identical to the amino acid sequence of SEQ ID NO:1, wherein the polynucleotide is a replicable polynucleotide encoding a collagen-like protein and wherein the amino acid sequence comprises a deletion of at least 38 amino acids at the N-terminus of the amino acid sequence of SEQ ID NO:1.

It is preferred, when the amino acid sequence comprises a deletion of between 38 and 74 amino acids at the N-terminus of the amino acid sequence of SEQ ID NO:1. This includes a complete deletion of the N-terminal V-domain (comprising 74 amino acids) and different truncations of the V-domain of at least 38 amino acids.

In a preferred embodiment, the amino acid sequence that is at least ≥60%, identical to the amino acid sequence of SEQ ID NO:1, SEQ ID NO:2, SEQ ID NO:3 or SEQ ID NO:4.

In a further configuration, the amino acid sequence that is at least ≥65%, or ≥70%, or ≥75%, or ≥80%, or ≥85% identical to the amino acid sequence of SEQ ID NO:2, SEQ ID NO:3 or SEQ ID NO:4.

In a preferred configuration, the polynucleotide encodes an amino acid sequence that is at least ≥90%, ≥92%, ≥94%, ≥96%, ≥97%, ≥98%, ≥99% or 100%, preferably ≥97%, particularly preferably ≥98%, very particularly preferably ≥99%, and extremely preferably 100%, identical to the amino acid sequence of SEQ ID NO:2, SEQ ID NO:3 or SEQ ID NO:4.

In a preferred embodiment of the present invention the polynucleotide is a replicable nucleotide sequence encoding the collagen-like protein from Streptococcus pyogenes.

Polynucleotide and nucleic acid molecules comprising such sequences and encoding polypeptide variants of SEQ ID NO:1 to 4, which contain one or more insertion(s) or deletion(s) are suitable as well. Preferably, the polypeptide contains a maximum of 5, a maximum of 4, a maximum of 3, or a maximum of 2, insertions or deletions of amino acids.

Mixture of polypeptides comprising one of the polypeptide variants of SEQ ID NO:1 to 4 and on or more of the truncated variants of the collagen-like protein of SEQ ID NO:5 to 12 can be used as well.

Plasmids and vectors that comprise the nucleotide sequences according to the invention and optionally replicate in microorganisms of the genera Pichia, Corynebacterium, Pseudomonas or Escherichia or are suitable. In a preferred configuration, the vector comprising the nucleotide sequences according to the present invention is suitable for replication in yeast of the genus Pichia pastoris.

Microorganisms of the genera Pichia, Corynebacterium, Pseudomonas or Escherichia that comprise the polynucleotides, vectors and polypeptides according to the invention are suitable as well. Preferred microorganisms are Pichia pastoris, Brevibacillus choshinensis or Corynebacterium glutamicum.

Microorganism of the species P. pastoris, E. coli, P. putida or C. glutamicum comprising any of the nucleotide sequences according to the present invention any of the polypeptides or any of the vectors according to the present invention are suitable.

The microorganism may be a microorganism in which the nucleotide sequence is present in overexpressed form.

Overexpression according to the invention means, generally, an increase in the intracellular concentration or activity of a ribonucleic acid, a protein (polypeptide) or an enzyme, compared with the starting strain (parent strain) or wild-type strain, if this is the starting strain. A starting strain (parent strain) is taken to mean the strain on which the measure leading to the overexpression was carried out.

In the overexpression, the methods of recombinant overexpression are preferred. These include all methods in which a microorganism is produced using a DNA molecule provided in vitro. Such DNA molecules comprise, for example, promoters, expression cassettes, genes, alleles, encoding regions etc. These are converted into the desired microorganism by methods of transformation, conjugation, transduction or like methods.

The extent of the expression or overexpression can be established by measuring the amount of the mRNA transcribed by the gene, by determining the amount of the polypeptide, and by determining the enzyme activity.

The bacterial collagen-like protein can be obtained in a fermentative process comprising the following steps:
a) fermentation of a microorganism according to the present invention in a medium,
b) accumulation of the bacterial collagen-like protein in the medium, wherein a fermentation broth is obtained.

The culture medium or fermentation medium that is to be used must appropriately satisfy the demands of the respective strains. Descriptions of culture media of various microorganisms are contained in the handbook “Manual of Methods for General Bacteriology” of the American Society for Bacteriology (Washington D.C., USA, 1981). The terms culture medium and fermentation medium or medium are mutually exchangeable.

As carbon source, sugars and carbohydrates can be used, such as, e.g., glucose, sucrose, lactose, fructose, maltose, molasses, sucrose-containing solutions from beet sugar or sugar cane processing, starch, starch hydrolysate and cellulose, oils and fats, such as, for example, soybean oil, sunflower oil, groundnut oil and coconut fat, fatty acids, such as, for example, palmitic acid, stearic acid and linoleic acid, alcohols such as, for example, glycerol, methanol and ethanol, and organic acids, such as, for example, acetic acid or lactic acid.

As nitrogen source, organic nitrogen compounds such as peptones, yeast extract, meat extract, malt extract, corn-steep liquor, soybean meal and urea or inorganic compounds such as ammonium sulphate, ammonium chloride, ammonium phosphate, ammonium carbonate and ammonium nitrate can be used. The nitrogen sources can be used individually or as a mixture.

As phosphorus source, phosphoric acid, potassium dihydrogenphosphate or dipotassium hydrogenphosphate or the corresponding sodium-containing salts can be used.

The culture medium must, in addition, contain salts, for example in the form of chlorides or sulphates of metals such as, for example, sodium, potassium, magnesium, calcium and iron, such as, for example, magnesium sulphate or iron sulphate, which are necessary for growth. Finally, essential growth substances such as amino acids, for example homoserine and vitamins, for example thiamine, biotin or pantothenic acid, can be used in addition to the above-mentioned substances.

Said starting materials can be added to the culture in the form of a single batch or supplied in a suitable manner during the culturing.

Basic compounds such as sodium hydroxide, potassium hydroxide, ammonia or ammonia water, or acid compounds such as phosphoric acid or sulphuric acid, are used in a suitable manner for pH control of the culture. The pH is generally adjusted to 6.0 to 8.5, preferably 6.5 to 8. For control of foam development, antifoams can be used, such as, for example, polyglycol esters of fatty acids. For maintaining the stability of plasmids, suitable selectively acting substances such as, for example, antibiotics, can be added to the medium. The fermentation is preferably carried out under aerobic conditions. In order to maintain said aerobic conditions, oxygen or oxygen-containing gas mixtures such as, for example, air, are introduced into the culture. The use of liquids that are enriched with hydrogen peroxide is likewise possible. Optionally, the fermentation is carried out at superatmospheric pressure, for example at a superatmospheric pressure of 0.03 to 0.2 MPa. The temperature of the culture is usually 20° C. to 45° C., and preferably 25° C. to 40° C., particularly preferably 30° C. to 37° C. In the case of batch or fed-batch processes, the culturing is preferably continued until an amount sufficient for the measure of obtaining the desired organic chemical compound has formed. This goal is usually reached within 10 hours to 160 hours. In continuous processes, longer culture times are possible. Due to the activity of the microorganisms, enrichment (accumulation) of the fine chemicals in the fermentation medium and/or in the cells of the microorganisms occurs.

Examples of suitable fermentation media may be found, inter alia, in patent documents U.S. Pat. Nos. 5,770,409, 5,990,350, 5,275,940, WO 2007/012078, U.S. Pat. No. 5,827,698, WO 2009/043803, U.S. Pat. No. 5,756,345 or U.S. Pat. No. 7,138,266; appropriate modifications may optionally be carried out to the requirements of the strains used.

The process may be characterized by a process which is selected from the group consisting of batch process, fed-batch process, repetitive fed-batch process and continuous process.

The process may be further characterized by a fine chemical, or a liquid, or a solid fine chemical-containing product is obtained from the fine chemical-containing fermentation broth.

The performance of the processes or fermentation processes according to the invention with respect to one or more of the parameters selected from the group of concentration (compound formed per volume), yield (compound formed per carbon source consumed), volumetric productivity (compound formed per volume and time) and biomass-specific productivity (compound formed per cell dry mass or bio dry mass and time or compound formed per cell protein and time) or other process parameters and combinations thereof, is increased by at least 0.5%, at least 1%, at least 1.5% or at least 2%, based on processes or fermentation processes with microorganisms in which the promoter variant according to the invention is present.

Owing to the measures of the fermentation, a fermentation broth is obtained which contains the desired collagen-like protein, and preferably amino acid or organic acid.

Then, a product in liquid or solid form that contains the collagen-like protein is provided or produced or obtained.

A fermentation broth means, in a preferred embodiment, a fermentation medium or nutrient medium in which a microorganism was cultured for a certain time and at a certain temperature. The fermentation medium, or the media used during the fermentation, contains/contain all substances or components that ensure production of the desired collagen-like protein and typically ensure growth and/or viability.

On completion of the fermentation, the resultant fermentation broth accordingly contains
a) the biomass (cell mass) of the microorganism resulting from growth of the cells of the microorganism,
b) the desired collagen-like protein formed in the course of the fermentation,
c) the organic by-products possibly formed in the course of the fermentation, and
d) the components of the fermentation medium used, or of the starting materials, that are not consumed by the fermentation, such as, for example, vitamins such as biotin, or salts such as magnesium sulphate.

The organic by-products include substances which are generated in addition to the respective desired compound by the microorganisms used in the fermentation and are possibly secreted.

The fermentation broth is withdrawn from the culture vessel or the fermentation container, optionally collected, and used for providing a product in liquid or solid form containing the collagen-like protein. The expression “obtaining the collagen-like protein-containing product” is also used therefor. In the simplest case, the collagen-like protein-containing fermentation broth withdrawn from the fermentation container is itself the product obtained.

By way of one or more of the measures selected from the group
a) partial (>0% to <80%) to complete (100%) or virtually complete (≥80%, ≥90%, ≥95%, ≥96%, ≥97%, ≥98%, ≥99%) removal of the water,
b) partial (>0% to <80%) to complete (100%) or virtually complete (≥80%, ≥90%, ≥95%, ≥96%, ≥97%, ≥98%, ≥99%) removal of the biomass, wherein this is optionally inactivated before the removal,
c) partial (>0% to <80%) to complete (100%) or virtually complete (≥80%, ≥90%, ≥95%, ≥96%, ≥97%, ≥98%, ≥99%, ≥99.3%, ≥99.7%) removal of the organic by-products formed in the course of the fermentation, and
d) partial (>0%) to complete (100%) or virtually complete (≥80%, ≥90%, ≥95%, ≥96%, ≥97%, ≥98%, ≥99%, ≥99.3%, ≥99.7%) removal of the components of the fermentation medium used or the starting materials that are not consumed by the fermentation,
a concentration or purification of the desired collagen-like protein is achieved from the fermentation broth. In this manner, products are isolated that have a desired content of the compound.

The partial (>0% to <80%) to complete (100%) or virtually complete (≥80% to <100%) removal of the water (measure a)) is also termed drying.

In a variant of the process, by complete or virtually complete removal of the water, the biomass, the organic by-products and the non-consumed components of the fermentation medium used, pure (≥80% by weight, ≥90% by weight) or high-purity (≥95% by weight, ≥97% by weight, ≥99% by weight) product forms of the desired collagen-like protein, preferably bacterial collagen-like protein, are successfully arrived at. For the measures according to a), b), c) or d), a great variety of technical instructions are available in the prior art.

In the case of processes for producing bacterial collagen-like protein processes are preferred in which products are obtained that do not contain any components of the fermentation broth. These products are used, in particular, in human medicine, in the pharmaceuticals industry, and in the food industry.

In the method the collagen like protein can preferably be present in the aqueous solution with a concentration range from 2.5 to 100 mg/ml.

In the method at least one cross linker is used, which undergoes a reaction with the at least one collagen-like protein via incubation to form the hydrogel.

In general, all cross-linker used in the field of collagen and collagen-like proteins are suitable.

In one embodiment the at least one cross-linker has at least two functional groups, which are able to undergo a reaction with the functional groups of the at least one collagen-like protein.

In one embodiment the at least one cross-linker is preferably selected from cross-linkers comprising at least two succinimidyl groups, 4-(4,6-dimethoxy-1,3,5-triazin-2-yl)-4-methyl-morpholinium chloride (DMTMM), glutaraldehyde, transglutaminase, diisocyanate, or a combination of 1-ethyl-3-(3-dimethylaminopropyl) carbodiimide (EDC) and N-hydroxysuccinimide (NHS). In a preferred embodiment the at least one cross-linker is selected from cross-linkers comprising at least two succinimidyl groups, 4-(4,6-dimethoxy-1,3,5-triazin-2-yl)-4-methyl-morpholinium chloride (DMTMM), transglutaminase, diisocyanate, or a combination of 1-ethyl-3-(3-dimethylaminopropyl) carbodiimide (EDC) and N-hydroxysuccinimide (NHS).

In a preferred embodiment the at least one cross-linker has one of following formulae (I) or (II):

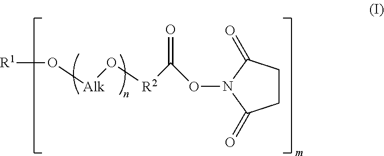

wherein
R¹ is a linear or branched alkyl group having up to 12 carbon atoms, preferably up to 8 carbon atoms, more preferably having five carbon atoms;
Alk is —CH₂—, —CH₂—CH₂— or —CH₂—CH₂—CH₂—, preferably —CH₂—CH₂—;
n is an integer from 1 to 1350, preferably, 50 to 1000, more preferably 125 to 660;
R² is —CH₂—, —C₂H₄—NH—(C═O)—C₃H₆—, —C₂H₄—O—, —C₂H₄—O—(C═O)—C₃H₆—, —C₂H₄—NH—(C═O)—C₂H₄— or —C₂H₄—O—(C═O)—C₂H₄—;
m is an integer from 2 to 8, preferably 4 to 8, more preferably 4;
or

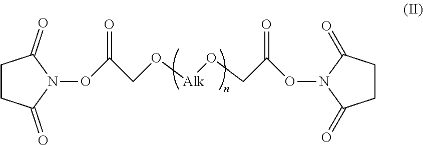

wherein
Alk is —CH₂—, —CH₂—CH₂— or —CH₂—CH₂—CH₂—, preferably —CH₂—CH₂—; and
n is an integer from 1 to 1350, preferably 50 to 1000, more preferably 125 to 660.

In one embodiment the cross-linker has following formula (III)

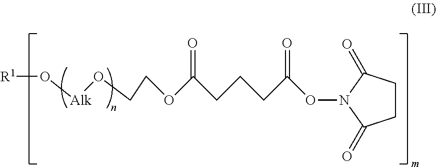

wherein
R¹ is a linear or branched alkyl group having up to 12 carbon atoms, preferably up to 8 carbon atoms, more preferably having five carbon atoms, most preferably is

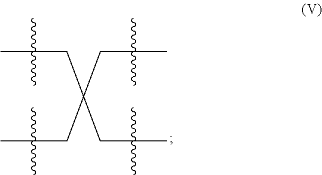 [SMILES: *CC(C*)(C*)C*]

Alk is —CH₂—, —CH₂—CH₂— or —CH₂—CH₂—CH₂—, preferably —CH₂—CH₂—;
n is an integer from 1 to 1350, preferably, 50 to 1000, more preferably 125 to 660;
m is an integer from 2 to 8, preferably 4 to 8, most preferably 4.

In one embodiment the cross-linker has a molecular weight of 1.000 to 60.000 g/mol, preferably 2.000 to 40.000 g/mol.

In one embodiment the at least one cross-linker is provided in an aqueous solution having a pH value of 6 to 8, preferably 6.8 to 7.4.

In one embodiment the functional groups of the at least one collagen-like protein to the functional groups of the at least one cross-linker, which undergo a reaction with each other, are present in a ratio of 1:0.01 to 1 to 5.

In the method according to the present invention at least one additive can be optionally present. The at least one additive can be present in step i) and/or step v). If additive is added in both steps i) and v) the same additive or different additives can be added.

In one embodiment the at least one additive is a growth factor, for example a fibroblast growth factor, epidermal growth factor, nerve growth factor or connective tissue growth factor or a recombinant human bone morphogenesis protein.

In one embodiment the at least one additive is selected from thrombin, fibrinogen, chitosan, silicic acid precursors, heparin, heparin derived oligosaccharides, hyaluronic acid, and glycosaminoglycans.

In one embodiment of the method, the incubation is performed for 5 mins to 48 h and/or at 4 to 37° C.

In one embodiment of the method, the buffer has an pH value of from 5.5 to 8.2, and/or is selected from 2-[4-(2-hydroxyethyl) piperazin-1-yl]ethane-1-sulfonic acid buffer, 2-morpholin-4-ylethanesulfonic acid buffer; and phosphate-buffered saline.

In one embodiment of the method, the lyophilization step is performed at −40 to −60° C.; and/or the obtained hydrogel is cooled to −20 to −80° C. before the lyophilization step is performed.

By performing the method according to the invention, a sponge is obtained.

In one embodiment the sponge has a water uptake capacity of 800 to 3000%, based on the total dry weight of the sponge.

In one embodiment the sponge has a pore size of 15 to 300 μm.

In one embodiment the sponge has a total porosity of 75 to 99%, and an open porosity of 20 to 85%.

In one embodiment the sponge has a Young's modulus of 45 to 250 kPa in dry form.

In one embodiment the sponge has a Young's modulus of 4 to 35 kPa in wet form.

The above-mentioned characteristics of the sponge are determined as described in the example section.

The sponge of the present invention can be used for wound sealing, haemostasis, wound plugging, healing promotion, bone regeneration, cartilage repair, cell cultures, production of vegetarian or vegan meat, the absorption of biological fluids, like blood or wound exudate.

### Protein Sequences

SEQ ID NO:1 Streptomyces pyogenes Collagen-like protein (CLP), full length protein
SEQ ID NO:2 Streptomyces pyogenes CLP, truncation 3
SEQ ID NO:3 Streptomyces pyogenes CLP, truncation 5
SEQ ID NO:4 Streptomyces pyogenes CLP, no V-domain

### EXAMPLES

In the examples, collagen-like protein was obtained according to the following method and is referred to as “rCol” as well.

### Production of Collagen-Like Protein

The bacterial collagen-like protein was produced in different host cells by fermentation.

To produce Scl2 from Streptomyces pyogenes in Pichia pastoris, the sequence of the collagen domain of the gene scl2, encoding for a collagen-like protein, has been codon optimized using different algorithms, and cloned in a secretion vector for Pichia pastoris and transformed in Pichia pastoris following standard protocol and subsequent application of a standard expression protocol in fed-batch mode, protein corresponding to Scl2p was detected in the supernatant of cell culture. (Damasceno, L. M., Huang, C J. & Batt, C. A. Protein secretion in Pichia pastoris and advances in protein production. Appl Microbiol Biotechnol 93, 31−39 (2012)).

Upon fermentation, supernatant has been separated from biomass via centrifugation (12000 g, 5 mins at room temperature).

The protein could be produced under similar conditions using either E. coli or C. glutamicum. In case of a production in yeast or C. glutamicum, the CL single strand is secreted by the cell. No cell lysis is needed as an initial purification step in this approach. In case of a production in E. coli a cell lysis is mandatory to remove the product from the cell.

The full-length collagen-like protein, a truncated variant (truncation 3) and the no-V-domain variant (based on the gene scl2 from Streptomyces pyogenes) were also expressed in Brevibacillus choshinensis. Therefore, the corresponding DNA sequences were cloned into a suitable secretion vector for B. choshinensis. Transformation of B. choshinensis with the new constructed plasmids was done according to Mizukami et al. 2010 (Curr Pharm Biotechnol 2010, 13:151−258).

The B. choshinensis strains were analyzed for their ability to produce the different collagen proteins in batch cultivations at 33° C. and pH 7 using the DASGIP® parallel bioreactor system from Eppendorf (Hamburg, Germany). The fermentation was performed using 1 L reactors. The production medium (TM medium, Biomed Res Int 2017, 2017:5479762) contained 10 g/L glucose. Upon fermentation, supernatant has been separated from biomass by centrifugation and was used for SDS PAGE analysis. For all three variants, collagen-like protein was produced.

The full-length collagen-like protein and the no-V-domain variant (based on the gene scl2 from Streptomyces pyogenes) were also expressed in Corynebacterium glutamicum. Therefore, the corresponding DNA sequences were cloned together with an upstream located signal peptide for protein secretion into a shuttle vector for C. glutamicum (Biotechnology Techniques 1999, 13:437-441.). The C. glutamicum strain ATCC 13032 was transformed with the new constructed plasmids by means of electroporation as described by Ruan et al. (Biotechnology Letters 2015, 37:2445-2452).

The C. glutamicum strains were analysed for their ability to produce the different collagen proteins in fed-batch cultivations at 30° C. and pH 7 using the DASGIP® parallel bioreactor system from Eppendorf (Hamburg, Germany). The fermentation was performed using 1 L reactors. The production medium contained 20 g/L glucose in the batch phase and the fed-batch phase was run with a glucose feed of 4 g/L*h. Upon fermentation, supernatant has been separated from biomass by centrifugation and was used for HPLC analysis. For both variants, collagen protein was produced. For the truncated variant of the collagen-like protein, product titer was higher as for the full-length variant.

After cell separation (via centrifugation) and folding of the bacterial collagen-like protein (via cooling of the concentrate) the bacterial collagen-like protein was purified using precipitation with 2-Propanol at 15 v %. After precipitation of the Scl2 protein a centrifugation was performed. The pellet was dissolved in water, the triple helical Scl2 protein was unfolded at 40° C. and filtered through a 100 kD membrane. This step serves to remove large sized impurities. The collected permeate was then concentrated in the consecutive 10 kD filtration. The retentate was washed to remove small sized impurities.

By that means a triple helical Scl2 protein purity >75 w % was achieved.

In the comparative examples animal derived collagen, i.e., rat tail collagen, commercially available from Sigma Aldrich under product number C7661 is used and is referred to as “rtCol” as well.

### Example 1: Sponges Made from Collagen-Like Protein Cross-Linked with EDC/NHS

Sponges were made by cross-linking of collagen-like protein with 1-Ethyl-3-(3-dimethylaminopropyl) carbodiimide (EDC) and N-Hydroxysuccinimide (NHS), followed by lyophilizing of resultant hydrogels. This technology is based on the reaction of EDC with carbon acid side chains of collagen's amino acids, such as aspartic acid and glutamic acid, forming active esters. The coupled EDC is replaced by NHS, increasing the cross-linking efficiency, and decreasing unspecific side reactions. Resulting NHS-ester in turn reacts with primary amines of collagen's lysine side chains. Thereby, several collagen molecules are covalently linked to form a three-dimensional protein scaffold. Here, final concentrations of 10, 20, and 40 mg/ml collagen-like protein were cross-linked with a molar ratio of collagen's carboxylic side chains to cross-linker of 1:1:1. Rat tail collagen in a concentration of 5 mg/ml was cross-linked in equivalent molar ratio to compare collagen-like protein with an animal-derived reference collagen.

For the primary gelation, the following reagents and solutions are required:
a. 100 mg/ml collagen-like protein dissolved in ultrapure H₂O
b. 6.3 mg/ml rat tail collagen (Sigma Aldrich, product number C7661) dissolved in 0.1% acetic acid
c. 1-Ethyl-3-(3-dimethylaminopropyl)carbodiimid-hydrochloride (EDC*HCl; Roth, product number 2156.2)
   for a 1532.4 mM stock solution, 294 mg/ml prepared in ultrapure H₂O
   for a 244.6 mM stock solution, 46.9 mg/ml dissolved in ultrapure H₂O
d. N-hydroxy succinimide (NHS; Sigma Aldrich, product number 130672)
   for a 1532.4 mM solution, 176 mg/ml dissolved in ultrapure H₂O
   for a 244.6 mM solution, 28.2 mg/ml prepared in ultrapure H₂O
c. for a 0.1 M washing solution, 1.4 g disodium hydrogen phosphate (Na₂HPO₄; Merck, product number #1.06586) dissolved in 100 ml ultrapure H₂O
d. ddH₂O

In detail, a collagen-like protein stock solution of 100 mg/ml was prepared in ultrapure water, and homogenized overnight by orbital shaking with a speed of 450 rpm. Under the same conditions, rat tail collagen was dissolved at a concentration of 6.3 mg/ml in 0.1% acetic acid. Before further processing, occurring air bubbles trapped in the collagen stock solutions were removed by centrifugation for at least 5 min at 250×g₀. On the day of use, the EDC*HCl and NHS cross-linker solutions (applied concentrations are described in Table 1 to 4) were freshly prepared in ultrapure water, and homogenously dissolved by vortexing. Collagen stock solutions were diluted with respective amount of water, subsequently adding EDC*HCl and NHS stock solutions (for pipetting scheme see Table 1 to 4) to finally obtain a gelation formulation. From that mixture, a volume of 1 ml was filled in a 24-well silicone mold, which results in a filling height of 5 mm for each sponge.

TABLE 1
Pipetting scheme for sponges made from collagen-like
protein (rCol) with a concentration of 10, 20, 40
mg/ml, and cross-linked with a molar ratio of collagen's
carboxylic groups to EDC and NHS of 1:1:1.

| | final | | stock | dilution | volume |
|---|---|---|---|---|---|
| | concentration | unit | solution | factor | [μl] |
| rCol | 10 | mg/ml | 100 | 10 | 100 |
| EDC*HCl | 15 | mM | 1532.4 | 100 | 10 |
| NHS | 15 | mM | 1532.4 | 100 | 10 |
| H₂O | NA | NA | NA | NA | 880 |
| rCol | 20 | mg/ml | 100 | 5 | 200 |
| EDC*HCl | 31 | mM | 1532.4 | 50 | 20 |
| NHS | 31 | mM | 1532.4 | 50 | 20 |
| H₂O | NA | NA | NA | NA | 760 |
| rCol | 40 | mg/ml | 100 | 2.5 | 400 |
| EDC*HCl | 61 | mM | 1532.4 | 25 | 40 |
| NHS | 61 | mM | 1532.4 | 25 | 40 |
| H₂O | NA | NA | NA | NA | 520 |

TABLE 2
Pipetting scheme for sponges made from rat tail collagen (rtCol)
with a concentration of 5 mg/ml, and cross-linked with a molar
ratio of collagen's carboxylic groups to EDC and NHS of 1:1:1.

| | final | | stock | dilution | volume |
|---|---|---|---|---|---|
| | concentration | unit | solution | factor | [μl] |
| rtCol | 5 | mg/ml | 6.3 | 1.25 | 800 |
| EDC*HCl | 5 | mM | 244.6 | 50 | 20 |
| NHS | 5 | mM | 244.6 | 50 | 20 |
| H₂O | NA | NA | NA | NA | 160 |

After approximately 2 hours, the newly formed hydrogels were covered with parafilm, to prevent dry out. For complete gelation, the hydrogels were stored overnight at room temperature. Subsequently, collagen's remaining activated groups were deactivated by washing with 0.1 M Na₂HPO₄ for 1 hour. Following, the hydrogels were washed with ultrapure water for one hour thrice and once overnight, orbital shaking with a speed of 200 rpm. After washing, the hydrogels were lyophilized using a Christ LSC Plus vacuum drying machine. A process defined for collagen samples was applied (for details see Table 5). First, the hydrogels were frozen with a speed of −1° C. per minute to −45° C. The frozen hydrogels were then dried under a vacuum of 0.07 mbar, and at a starting temperature of −30° C., subsequently increasing the temperature to 20° C.

TABLE 5
Drying process applied for sponges made
by lyophilization of hydrogels.

| | Temperature | Pressure | Time |
|---|---|---|---|
| | [° C.] | [mbar] | [h] |
| loading | 15 | — | — |
| freezing | 15 | — | 0.5 |
| | −45 | — | 1.0 |
| | −45 | — | 2.0 |
| main drying | −45 | 0.07 | 1.0 |
| | −30 | 0.07 | 1.0 |
| | −30 | 0.07 | 5.0 |
| | −25 | 0.07 | 5.0 |
| | −20 | 0.07 | 5.0 |
| | −15 | 0.07 | 5.0 |
| | −10 | 0.07 | 5.0 |
| | −5 | 0.07 | 5.0 |
| | 0 | 0.07 | 2.0 |
| | 5 | 0.07 | 2.0 |
| final drying | 20 | 0.001 | 1.0 |
| | 20 | 0.001 | 1.0 |

### Example 2: Sponges Made from Collagen-Like Protein Cross-Linked with 4-Arm-PEG-SG

Collagen sponges were manufactured by cross-linking of collagen-like protein with 4-Arm-PEG-SG. The applied cross-linker is a multi-arm PEG derivative with a pentaerythritol core and four terminal N-Hydroxysuccimidyl moieties (NHS). In a substitution reaction, those NHS groups are replaced by stable amide bonds formed from primary amines with collagen's lysine side chains. By connecting several collagen molecules, gelation occurs. Resultant hydrogels are subsequently lyophilized to obtain sponges.

For comparison of different cross-linking technologies, sponges were made with collagen-like protein (10, 20, and 40 mg/ml), as well as with rat tail collagen (5 mg/ml) as described in Example 1. In this example, a molar ratio of collagen's lysine residues to 4-Arm-PEG-SG of 1:0.05 was applied. Furthermore, two 4-Arm-PEG-SG derivates with a varying core size unit, resulting in a total mass of 10 kDa or 40 kDa, were tested.

In addition to the collagen solutions mentioned in Example 1, the following reagents are required:
a. 4-arm PEG Succinimidyl Glutarate (4-Arm-PEG-SG)
   10 kDa derivate (JenKem Technology USA, product number A7031-1/4ARM-SG-10K, M: 10000 g/mol)
      for a 9.6 mM solution, 96 mg/ml dissolved in ultrapure H₂O
      for a 2.1 mM solution, 21 mg/ml prepared in ultrapure H₂O
   40 kDa derivate (JenKem Technology USA, product number A7017-1/4ARM-SG-40K, M: 40000 g/mol)
      for a 9.6 mM solution, 384 mg/ml dissolved in ultrapure H₂O
      for a 2.1 mM solution, 84 mg/ml prepared in ultrapure H₂O
b. 1 M HEPES-buffer of pH 8.0
   23.83 g HEPES dissolved in 80 ml ultrapure H₂O, adjusted with 1 M NaOH to pH 8.0, and added ultrapure H₂O until 100 ml
c. ddH₂O

As mentioned in Example 1, a collagen-like protein solution with a concentration of 100 mg/ml was prepared in ultrapure water, and a rat tail collagen solution with a concentration of 6.3 mg/ml was dissolved in 0.1% acetic acid. A 1 M HEPES solution of pH 8.0 was applied to improve gelation efficiency at basic pH. The 4-Arm-PEG-SG stock solutions were freshly prepared in ultrapure water. Step-by-step collagen solution, 1 M HEPES buffer of pH 8.0 and water were mixed, succeeding with the addition of the cross-linker solution (for details of the pipetting scheme see Table 6 for sponges made from collagen-like protein and Table 7 for reference collagen).

TABLE 6
Pipetting scheme for a gelation solution made from collagen-
like protein (rCol) with a concentration of 10, 20 and
40 mg/ml, and cross-linked with a molar ratio of collagen's
lysine residues to 4-Arm-PEG-SG of 1:0.05.

| | final | | stock | dilution | volume |
|---|---|---|---|---|---|
| | concentration | unit | solution | factor | [μl] |
| rCol | 10 | mg/ml | 100 | 10 | 100 |
| PEG | 0.5. | mM | 9.6 | 20 | 50 |
| HEPES | 100 | mM | 1000 | 10 | 100 |
| H₂O | NA | NA | NA | NA | 750 |
| rCol | 20 | mg/ml | 100 | 5 | 200 |
| PEG | 1 | mM | 9.6 | 10 | 100 |
| HEPES | 100 | mM | 1000 | 10 | 100 |
| H²O | NA | NA | NA | NA | 600 |
| rCol | 40 | mg/ml | 100 | 2.5 | 400 |
| PEG | 2 | mM | 9.6 | 5 | 200 |
| HEPES | 100 | mM | 1000 | 10 | 100 |
| H₂O | NA | NA | NA | NA | 300 |

TABLE 7
Pipetting scheme for a gelation solution made from rat tail collagen
(rtCol) with a concentration of 5 mg/ml, and cross-linked with
a molar ratio of lysine residues to 4-Arm-PEG-SG of 1:0.05.

| | final | | stock | dilution | volume |
|---|---|---|---|---|---|
| | concentration | unit | solution | factor | [μl] |
| rtCol | 5 | mg/ml | 6.25 | 1.25 | 800 |
| PEG | 0.1 | mM | 2.1 | 20 | 50 |
| HEPES | 100 | mM | 1000 | 10 | 100 |
| H₂O | NA | NA | NA | NA | 50 |

After homogenous mixing, a volume of 1 ml gelation formulation was filled in 24-well silicone molds. After a very fast gelation within minutes, the hydrogels were covered with parafilm, and stored overnight at room temperature. On the next day, the hydrogels were washed with ultrapure water for one hour thrice and once overnight, orbitally shaking with a speed of 200 rpm. After washing, the hydrogels were lyophilized with the process described in Example 1.

### Example 3: Sponges Made from Collagen-Like Protein Cross-Linked with DMTMM

A further technology to manufacture sponges by cross-linking the collagen-like protein is based on 4-(4,6-Dimethoxy-1,3,5-triazin-2-yl)-4-methylmorpholinium chloride (DMTMM). DMTMM activates collagen's carbon acid side chains, such as aspartic acid and glutamic acid, and forms active esters. The resulting highly reactive ester enables a nucleophilic attack by primary amines of collagen's lysine side chains. This nucleophilic substitution releases 4,6-dimethoxy-1,3,5-triazin-2-ol, and finally forms an amide bond.

As applied for the other cross-linking technologies, sponges were made from collagen-like protein in concentrations of 10, 20 and 40 mg/ml. Consistently, sponges were manufactured with rat tail collagen in a concentration of 5 mg/ml. Here, a molar ratio of collagen's carboxylic groups to DMTMM of 1:0.4 was applied.

For this technology, the following specific reagents and solutions are required:
a. 4-(4,6-Dimethoxy-1,3,5-triazin-2-yl)-4-methylmorpholinium chloride (DMTMM; TCI chemicals, product number D2919)
   for 245.2 mM solution, 68 mg/ml prepared in ultrapure H₂O
   for 97.9 mM solution, 27 mg/ml dissolved in ultrapure H₂O
b. ddH₂O

A collagen-like protein solution with a concentration of 100 mg/ml and a rat tail collagen solution with a concentration of 6.3 mg/ml were prepared as mentioned before (see Example 1). The DMTMM cross-linker was freshly dissolved in ultrapure water. The collagen stock solution and water were premixed before adding the DMTMM cross-linker solution (for details see pipetting scheme in Table 8 and Table 9).

TABLE 8
Pipetting scheme for sponges made from collagen-like
protein (rCol) with a concentration of 10, 20, and
40 mg/ml, and cross-linked with a molar ratio of
collagen's carboxylic groups to DMTMM of 1:0.4.

| | final | | stock | dilution | volume |
|---|---|---|---|---|---|
| | concentration | unit | solution | factor | [μl] |
| rCol | 10 | mg/ml | 100 | 10 | 100 |
| DMTMM | 6 | mM | 245.2 | 40 | 25 |
| H₂O | NA | NA | NA | NA | 875 |
| rCol | 20 | mg/ml | 100 | 5 | 200 |
| DMTMM | 12 | mM | 245.2 | 20 | 50 |
| H₂O | NA | NA | NA | NA | 750 |
| rCol | 40 | mg/ml | 100 | 2.5 | 400 |
| DMTMM | 25 | mM | 245.2 | 10 | 100 |
| H₂O | NA | NA | NA | NA | 500 |

TABLE 9
Pipetting scheme for sponges made from rat tail collagen (rtCol)
with a concentration of 5 mg/ml, and cross-linked with a molar
ratio of collagen's carboxylic groups to DMTMM of 1:0.4.

| | final | | stock | dilution | volume |
|---|---|---|---|---|---|
| | concentration | unit | solution | factor | [μl] |
| rtCol | 5 | mg/ml | 6.25 | 1.25 | 800 |
| DMTMM | 2 | mM | 97.9 | 50 | 20 |
| H₂O | NA | NA | NA | NA | 180 |

After thoroughly mixing all components, a volume of 1 ml gelation solution was filled in 24-wells of a silicone mold. Gelation starts within 20 minutes, and thereafter, the mold was covered with parafilm and stored at room temperature overnight. On the next day, the hydrogels were washed with ultrapure water for one hour thrice and once overnight, orbitally shaking with a speed of 200 rpm. After washing, the hydrogels were lyophilized as described in Example 1.

Fluid Absorption, Shrinkage and Swelling Behavior of Sponges Made from Collagen-Like Protein

The fluid absorption was calculated from weight measurements of the sponges after lyophilization, and after submersion in phosphate buffered saline. For wet weight assessment, the sponges were submersed in 1 ml phosphate buffered saline. After incubation for 24 h at room temperature, the buffer solution and remaining excess liquid were removed, and the wet weight was measured. The fluid absorption is relatively described to dry weight values, as summarized by following equation:

$$Fluid adsorption [ \% ] = [ {weight}_{24 h} - {weight}_{dry} ] / {{weight}_{dry}}^{*} 100$$

The shrinkage and swelling of sponges were determined by the relative diameter change before and after submersion in phosphate buffered saline, as expressed in the equation below:

$$Diameter change [ \% ] = Δ {diameter}_{after submersion - before submersion} / {{diameter}_{before submersion}}^{*} 100$$

Pore Size, Total Porosity and Open Porosity of Sponges Made from Collagen-Like Protein

Sponges made from collagen-like protein with a concentration of 10, and 40 mg/ml and sponges made from rat tail collagen with a concentration of 5 mg/ml were frozen in liquid nitrogen and cut with a blade to create a brittle fracture cross-sectional surface. Sections were fixed with a sticky pad on a SEM sample stub. The sample surface was sputtered with gold-palladium, thereby increasing electrical conductivity. With a scanning electron microscope (SEM, high vacuum, 10 kV, SE-mode), secondary electron pictures were captured in various magnifications. The pore size of several cells was measured (n≥8) with an image analysis software.

The total porosity and the open porosity of the sponges were assessed by a liquid displacement with ethanol (99%). Therefore, the sponges' dry weight, and the wet weight, after submersing the sponge for one hour in ethanol, were assessed. Furthermore, the applied ethanol, and the remaining ethanol after lifting out the soaked sponge were weighted.

The total porosity, is calculated from the single measured values as described by equation below:

$$Total porosity [ \% ] = {EtOH}_{added} - {EtOH}_{remaining} {/ [ {Sponge}_{dry} + {EtOH}_{added} - {EtOH}_{remaining} ]}^{*} 100$$

The open porosity is assessed by the quotient of ethanol soaked by the sponge to the hypothetical weight of sponges' volume entirely filled with ethanol. Here for, the geometrical volume (Vₛ) was calculated by measured diameter and height. The weight of sponges' volume entirely filled with ethanol was derived by multiplication of the geometrical volume with the density of ethanol (ρₑ; 0.789 g/cm³). The calculation of open porosity is summarized as follows:

$$Open porosity [ \% ] = [ {Sponge}_{soaked} - {Sponge}_{dry} ] / ρ {e}^{*} {{V}_{s}}^{*} 100$$

Mechanical Characterization of Sponges Made from Collagen-Like Protein

A test setup was designed for compression of sponges in dry state and after incubation for 24 h in phosphate buffered saline. For the mechanical characterization, a CT3 texture analyzer from Brookfield with a maximum load of 4500 g was applied. This mechanical testing device was equipped with a probe (11.3 mm diameter, 1 cm² surface) equal or slightly smaller than the diameter of the test samples (approximately from 11 to 16 mm diameter). The compression was performed with a trigger point of 5 g and a measurement velocity of 0.5 mm/s. During the compression process, the applied weight and respective achieved distance were tracked. The recorded raw data was used to calculate the stress and the strain, finally plotting both values as a function of each other. From this stress-strain plot, the Young's modulus was derived as the slope in the linear elastic range. For dry sponges, the linear elastic range from 5 to 20% strain was included in the calculation. For wet sponges, the linear elastic range from 2 to 10% strain was applied for the calculation of Young's modulus. Here, exemplarily sponges made from 20 mg/ml collagen-like protein were compared to Lyostypt, a commercially available sponge from B. Braun (product number 1069128).

1. A method of preparing a sponge based on collagen-like protein, the method comprising:
i) providing an aqueous solution comprising at least one collagen-like protein and optionally at least one additive;
ii) cross-linking the at least one collagen-like protein with at least one cross-linker via incubation to obtain a hydrogel;
iii) optionally washing the hydrogel with a buffer;
iv) performing a lyophilization to obtain the sponge;
v) optionally adding at least one additive; and
vi) optionally sterilizing the obtained sponge.

2. The method according to claim 1, wherein the functional groups of the at least one collagen-like protein to the functional groups of the at least one cross-linker, which undergo a reaction with each other, are present in a ratio of 1:0.01 to 1:5.

3. The method according to claim 1, wherein the at least one collagen-like protein is
i) bacterial collagen-like protein; and/or
ii) is present in the aqueous solution with a concentration range from 2.5 to 100 mg/ml.

4. The method according to claim 1, wherein the at least one cross-linker
i) has at least two functional groups, which are able to undergo a reaction with the functional groups of the at least one collagen-like protein; and/or
ii) is selected from the group consisting of cross-linkers comprising at least two succinimidyl groups, 4-(4,6-dimethoxy-1,3,5-triazin-2-yl)-4-methyl-morpholinium chloride (DMTMM), glutaraldehyde, transglutaminase, diisocyanate, and a combination of 1-ethyl-3-(3-dimethylaminopropyl) carbodiimide (EDC) and N-hydroxysuccinimide (NHS); and/or
iii) is provided in an aqueous solution having a pH value of 6 to 8; and/or
iv) has a molecular weight of 1.000 to 60.000 g/mol.

5. The method according to claim 1, wherein an incubation is performed for 5 mins to 48 h and/or at 4 to 37° C.

6. The method according to claim 1, wherein the buffer has an pH value of from 5.5 to 8.2, and/or is at least one selected from the group consisting of 2-[4-(2-hydroxyethyl) piperazin-1-yl]ethane-1-sulfonic acid buffer, 2-morpholin-4-ylethanesulfonic acid buffer and phosphate-buffered saline.

7. The method according to claim 1, wherein the lyophilization is performed at −40 to −60° C.; and/or
the obtained hydrogel is cooled to −20 to −80° C. before the lyophilization is performed.

8. A sponge obtained by the method according to claim 1.

9. The sponge according to claim 8, having:
i) a water uptake capacity of 800 to 3000%, based on the total dry weight of the sponge; and/or
ii) a pore size of 15 to 300 μm; and/or
iii) a porosity of a total porosity of 75 to 99%, and an open porosity of 20 to 85%; and/or
iv) a Young's modulus of 45 to 250 kPa in dry form; and/or
v) a Young's modulus of 4 to 35 kPa in wet form.

10. A method, comprising:
applying the sponge according to claim 8,
wherein said method comprises at least one selected from the group consisting of wound sealing, haemostasis, wound plugging, healing promotion, bone regeneration, cartilage repair, cell cultures, production of vegetarian or vegan meat, or the absorption of biological fluids.

11. The method according to claim 10, wherein the biological fluids are blood or wound exudate.
